(19) United States
(12) Patent Application Publication
(10) Pub. No.: US 2005/0049295 A1
(43) Pub. Date: Mar. 3, 2005

(54) Process for the preparation of 5-(2-(4-(1,2-benzisothiazol-3-yl)-1piperazinyl) ethyl)-6-chloro-1, 3-dihydro-2h-indol-2-one hydrochloride (ziprasidone hydrochloride) and its intermediate

(75) Inventors: Manne Satyanarayana Reddy, Hyderabad (IN); Sundaram Venkatraman, Hyderabad (IN); Srinivasan Thirumalai Rajan, Hyderabad (IN); Sharat Pandurang Narsapur, Secunderabad (IN); Manoj Ramesh Kharkar, Hyderabad (IN); Surya Narayana Devarkonda, Hyderabad (IN); Yarraguntla Sesha Reddy, Prakasam District (IN); Rangineni Srinivasulu, Mahaboobnagar (IN); Deepak K. Shukla, Maharashtra (IN); Pushkar B. Lakhekar, Maharashtra (IN); Uppala Venkata Bhaskar Rao, Hyderabad (IN); Mummadi Venkatesh, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: DR. REDDY'S LABORATORIES LIMITED; DR. REDDY'S LABORATORIES, INC.

(21) Appl. No.: 10/868,506

(22) Filed: Jun. 14, 2004

(30) Foreign Application Priority Data
Jun. 12, 2003 (IN) 488/MAS/2003
Mar. 12, 2004 (IN) 222/CHE/2004

(51) Int. Cl. A61K 31/404; C07D 209/36

(52) U.S. Cl. 514/418; 548/484

(57) ABSTRACT

The present invention relates to improved processes for the preparation of 5-(2-(4-(1,2-benzisothiazol-3-yl)-1-piperazinyl) ethyl)-6-chloro-1,3-dihydro-2H-indol-2-one and its hydrochloride, which is known as Ziprasidone hydrochloride of Formula (I) and 5-(2-Chloro acetyl)-6-chloro oxindole of Formula (IV), which is an intermediate for the preparation of 5-(2-chloro ethyl)-6-chloro oxindole of Formula (V). Ziprasidone hydrochloride of Formula (I) of the present invention is depicted by the following structure.

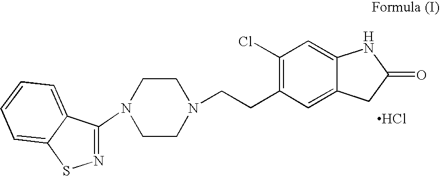

### FIELD OF THE INVENTION

The present invention relates to improved processes for the preparation of 5-(2-(4-(1,2-benzisothiazol-3-yl)-1-piperazinyl) ethyl)-6-chloro-1,3-dihydro-2H-indol-2-one and its hydrochloride, which is known as Ziprasidone hydrochloride of Formula (I) and 5-(2-Chloro acetyl)-6-chloro oxindole of Formula (IV), which is an intermediate for the preparation of 5-(2-chloro ethyl)-6-chloro oxindole of Formula (V). Ziprasidone hydrochloride of Formula (I) of the present invention is depicted by the following structure.

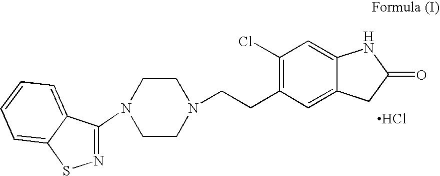

Ziprasidone, Ziprasidone hydrochloride and its other pharmaceutically acceptable salts are useful as antipsychotic agents in the treatment of Schizophrenia.

### BACKGROUND OF THE INVENTION

Several references disclosed the preparation of Ziprasidone, Ziprasidone hydrochloride salt; other salts including hydrochloride monohydrate, mesylate salts etc. U.S. Pat. No. 4,831,031 incorporated here in by reference, described the synthesis of Ziprasidone hydrochloride salt. It generically disclosed the preparation of 5-(2-Chloro acetyl)-6-chloro oxindole, which is an intermediate for the preparation of 5-(2-chloro ethyl)-6-chloro oxindole (one of the key intermediate of Ziprasidone) in presence of carbon disulphide.

The said patent also exemplified generically the coupling reaction between 3-(1-piperazinyl)-1,2-benzisothiazole and 5-(2-chloro ethyl)-6-chloro oxindole for the preparation of Ziprasidone base using polar solvents like lower alcohols, methylisobutylketone, and dichloromethane. The said patent also disclosed the preparation of Ziprasidone hydrochloride by adding aqueous hydrochloric acid to the Ziprasidone base.

U.S. Pat. No. 5,206,366, which also referred to an aqueous based process for preparing ziprasidone, its hydrochloride salt and no where disclosed the non-polar solvents for the coupling reaction between 3-(1-piperazinyl)-1,2-benzisothiazole and 5-(2-chloro ethyl)-6-chloro oxindole for the preparation of Ziprasidone base. U.S. Pat. No. 4,590,196 refers to 1-(1,2-benzisothiazol-3-yl)piperazine, which is the penultimate intermediate made by the processes of the present invention.

U.S. Pat. No. 5,312,925 disclosed preparation of the Ziprasidone hydrochloride monohydrate, which involves refluxing the reaction mixture of 3-(1-piperazinyl)-1,2-benzisothiazole hydrochloride, 5-(2-chloro ethyl)-6-chloro oxindole, water and sodium carbonate at 100° C., followed by isolating the required compound by washing with isopropanol and recrystallising from THF. One aspect of the present invention provides an improved process for the preparation of 5-(2-Chloro acetyl)-6-chloro oxindole, which is an intermediate for the preparation of 5-(2-chloro ethyl)-6-chloro oxindole (one of the key intermediate of Ziprasidone), Ziprasidone Base and its hydrochloride, which is cost effective, commercially viable and well suited for industrial scale up.

### SUMMARY OF THE INVENTION

The present invention relates to an improved, convenient, commercially viable, environment friendly and cost-effective processes for the preparation of 5-(2-Chloro acetyl)-6-chloro oxindole of Formula (IV), which is an intermediate for the preparation of 5-(2-chloro ethyl)-6-chloro oxindole of Formula (V) and 5-(2-(4-(1,2-benzisothiazol-3-yl)-1-piperazinyl) ethyl)-6-chloro-1,3-dihydro-2H-indol-2-one hydrochloride, which is known as Ziprasidone hydrochloride of Formula (I). The process for the preparation of 5-(2-Chloro acetyl)-6-chloro oxindole of Formula (IV) comprises the reaction of 6-Chloro-2-oxindole of Formula (II), chloroacetyl chloride of Formula (III) in presence of halogenated hydrocarbon solvents like dichloromethane and aluminum chloride as catalyst.

Another main aspect of the present invention is to provide an improved process for the preparation of Ziprasidone hydrochloride of Formula (I), which comprises of refluxing the reaction mixture of 3-(1-piperazinyl)-1,2-benzisothiazole of Formula (VI), 5-(2-chloro ethyl)-6-chloro oxindole of Formula (V) and sodium iodide in less polar solvents like cyclohexane in presence of a phase transfer catalyst like tetrabutyl ammonium bromide till the reaction completes. Then by subsequent steps washed the isolated compound with alkanone solvent like acetone.

It is still a further object of the present invention is to provide a process for purification of Ziprasidone base, which comprises of recrystalising the Ziprasidone base in a mixture of chloroform and methanol to yield the pure ziprasidone base, which is further acidified with HCl in acetic acid to yield the final ziprasidone hydrochloride salt.

Another aspect of the present invention provides processes for preparation and crystallization of ziprasidone hydrochloride, which directly produces large crystals of ziprasidone hydrochloride with greatly improved stability and purity. Preferably mean particle size of the ziprasidone hydrochloride crystals is greater than 85 micron.

Another aspect of the present invention also provides a packing for ziprasidone hydrochloride and its packing process.

The processes of the present invention are simple, cost-effective, and non-hazardous and are well suited for large-scale production.

### DETAILED DESCRIPTION OF THE INVENTION

The present invention relates to an improved process for the preparation of 5-(2-Chloro acetyl)-6-chloro oxindole of Formula (IV), which is an intermediate for the preparation of 5-(2-chloro ethyl)-6-chloro oxindole of Formula (V), includes:

i. refluxing the reaction mixture of 6-chloro-2-oxindole of Formula (II) and chloro acetyl chloride of Formula (III) in presence of halogenated hydrocarbon solvents like dichloromethane, chloroform, ethylene dichloride, carbon tetrachloride, preferably dichloromethane and Lewis acids like AlCl₃, BF₃. (Et₂O)₂, preferably AlCl₃ as catalyst till the reaction completes;

ii. cooling the reaction mass obtained in step (i) and hydrolysed into chilled water at a temperature of 15-45° C., preferably to 25-35° C. accompanied by stirring the reaction mass at the same temperature till the solid separates;

iii. successive filtering and washing the solid obtained in step (ii) with water;

iv. purifying 5-(2-Chloro acetyl)-6-chloro oxindole obtained by step (iii) or by different process in organic acids like acetic acid, formic acid, propionic acid preferably acetic acid by the process which comprises adding organic acid followed by heating the reaction mixture to a temperature of 60-100° C., preferably 70-80° C.;

v. adding activated carbon to the reaction mass of step (iv) and stirring at same temperature for 30 minutes, preferably 10-15 minutes;

vi. filtering the carbon from reaction mass obtained in step (v);

viii. maintaining the filtrate obtained in step (vi) to a temperature of 10-40° C., preferably 18-22° C. till the solid separates;

viii. filtering and washing the solid obtained in step (vii) with water;

ix. drying the solid obtained step (viii) to a temperature of 50-100° C., preferably 60-80° C. to afford pure 5-(2-Chloro acetyl)-6-chloro oxindole of Formula (IV).

Accordingly the other aspect of the present invention is to provide an improved process for the preparation of 5-(2-(4-(1,2-benzisothiazol-3-yl)-1-piperazinyl) ethyl)-6-chloro-1,3-dihydro-2H-indol-2-one hydrochloride of Formula (I) (Ziprasidone hydrochloride) includes;

A) Preparation of Ziprasidone Base of Formula (VII)
i. carrying out the reaction between the 3-(1-piperazinyl)-1,2-benzisothiazole of Formula (VI), 5-(2-chloro ethyl)-6-chloro oxindole of Formula (V), sodium carbonate and sodium iodide in a less polar solvents such as cyclohexane, toluene, benzene, hexanes, heptane preferably Cyclohexane in presence of a phase transfer catalysts like tetrabutyl ammonium bromide, tetrabutyl phosphonim bromide, tetraethyl ammonium bromide, tetrabutyl ammonium iodide preferably tetrabutyl ammonium bromide till the reaction completes at reflux or pressure conditions for example using autoclave.
ii. cooling the reaction mass obtained in step (i) to a temperature of 15-40° C., preferably to 25-35° C.;
iii. filtering the compound obtained in step (ii);
iv. adding water to the wet compound obtained in step (iii) accompanied by stirring;
v. washing the compound obtained in step (iv) with water;
vi. suspending the compound obtained in step (v) in alkanone solvents like acetone, 2-butanone, propanone, methylethyl ketone, dimethyl ketone, diethyl ketone, preferably acetone accompanied by stirring;
viii. isolating the solid obtained in step (vi);
viii. drying the isolated compound of step (vii) at 50-100° C., preferably 70-75° C. to afford the Ziprasidone base of Formula (VII);

B) Purification of Ziprasidone Base
i. dissolving the Ziprasidone Base of Formula (VII) in mixture of alcoholic and halogenated hydrocarbon solvents like methanol, ethanol, propanol, butanol, isopropanol, isobutanol, preferably methanol and halogenated hydrocarbon solvents like methylene chloride, chloroform, ethylene dichloride, carbon tetrachloride, preferably chloroform at reflux temperature;
ii. charging carbon to the reaction solution of step (i) and followed by maintaining at reflux condition for 30 minutes, preferably 10-15 minutes;
iii. filtering the carbon from reaction mass obtained in step (ii);
iv. optionally distilling off the solvent from the filtrate of step (iii) to about half of the volume;
v. adding alcoholic solvents like methanol, ethanol, propanol, butanol, isopropanol, isobutanol, preferably methanol to the residue of step (iv) at a temperature of 20-60° C. for 30 minutes, preferably 10-15 minutes;
vi. cooling the reaction mass of step (v) to of 0-40° C., preferably to 25-35° C. accompanied by maintaining the reaction mass at the same temperature till the solid separates out;
vii. filtering and washing the solid obtained in step (vi) with alcoholic solvents like methanol, ethanol, propanol, butanol, isopropanol, isobutanol, preferably methanol;
viii. drying the isolated compound of step (vii) at 50-100° C., preferably 65-75° C. to afford Ziprasidone base of Formula (VIII);

C) Preparation of Ziprasidone Hydrochloride of Formula (I)
i. dissolving Ziprasidone base of Formula (VIII) in organic acids like acetic acid, formic acid, preferably acetic acid or a mixture of organic acids and alcohols at room temperature;
ii. charging carbon to the reaction solution of step (i) accompanied by stirring for 30 minutes, preferably 10-15 minutes;
iii. filtering the carbon from reaction mass obtained in step (ii) accompanied by adding hydrochloric acid in organic solvents like acetic acid, isopropanol, methanol, preferably HCl in acetic acid or HCl in isopropanol to the filtrate or alternatively adding the solution obtained step (ii) to the mixture of hydrochloric acid in organic solvents like acetic acid, Isopropanol or mixture of acetic acid and isopropanol;
iv. maintaining the reaction mixture at 30-35° C. till the solid separates out;
v. isolating the solid obtained in step (iv);
vi. optionally slurrying the isolated compound from alcoholic solvents like methanol, ethanol, propanol, butanol, isopropanol, isobutanol, preferably isopropanol;
vii. drying the solid obtained in step (vi) at 50-100° C., preferably 70-80° C. to afford ziprasidone hydrochloride of Formula (I).

An improved process for the preparation of Ziprasidone hydrochloride of the present invention by above-disclosed method can be depicted by the following synthetic scheme.

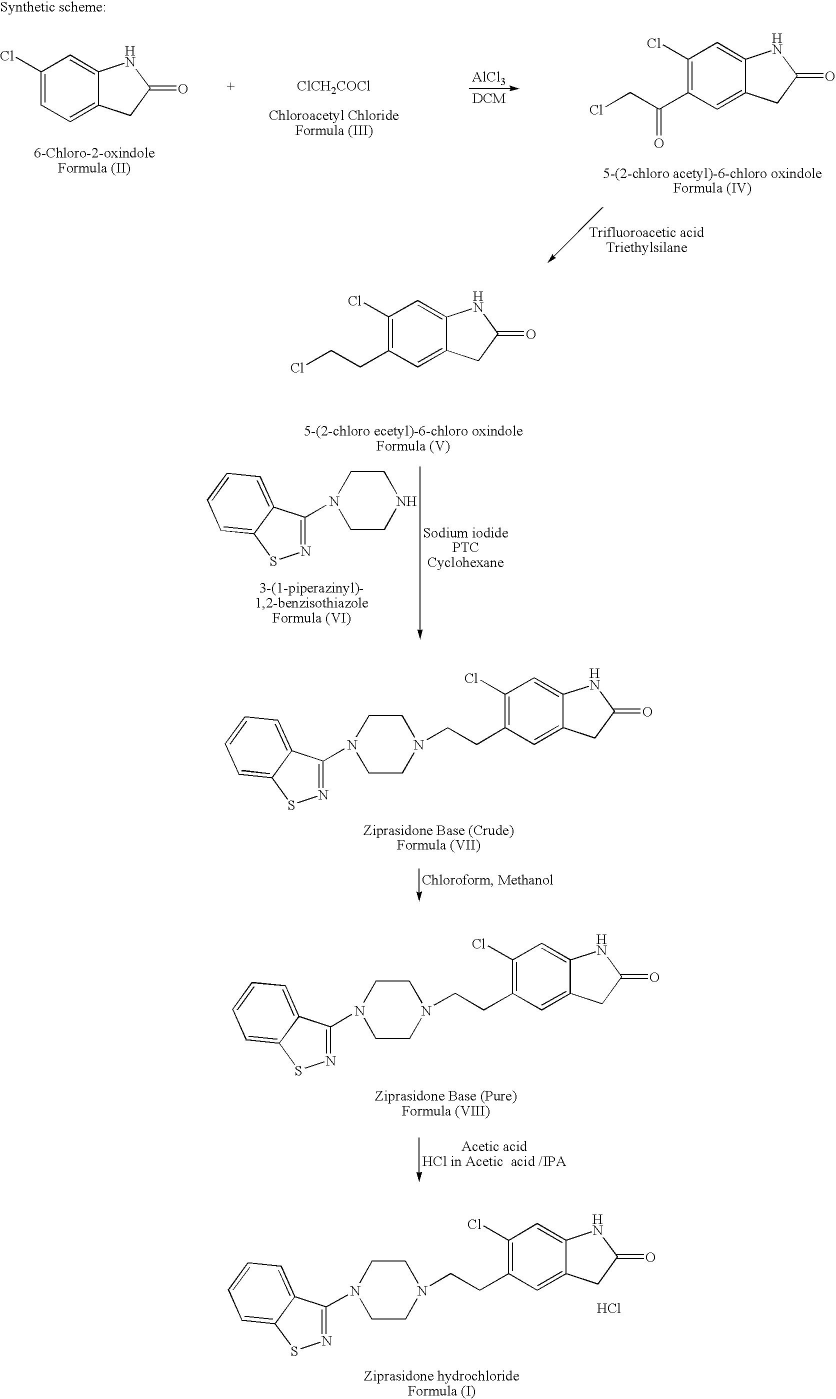

Thus, the present invention is directed to an improved process for the preparation of ziprasidone hydrochloride, which renders it well suited for pharmaceutical formulations.

Another embodiment of the present invention provides a process for the crystallization of ziprasidone hydrochloride, which includes:

a) dissolving Ziprasidone hydrochloride in carboxylic acid solvents like formic acid, at 20° C. to its reflux temperature of the solvent, preferably at 40 to 75° C.

b) optionally adding Ziprasidone hydrochloride seed compound at a temperature range between about 45 to 70° C.

c) maintaining the temperature of the mixture at 40-50° C. for two hours

d) cooling the mixture to between about 25 to 35° C.

e) adding antisolvent like ethyl acetate over a period of 30 to 60 minute at about 30° C.

f) isolating the compound by filtration

g) adding carboxylic acid solvent like acetic acid to the wet compound

h) stirring the mixture

i) filtering and washing with antisolvent like ethyl acetate

j) drying of the compound with or without vacuum at temperature of 20° C. to 130° C.

The present invention also provides the process for the crystallization of ziprasidone hydrochloride, which directly produces large crystals of ziprasidone hydrochloride including:

a) dissolving Ziprasidone hydrochloride in carboxylic acid solvents like formic acid at 10 to 45° C. and stirring till clear dissolution

b) heating antisolvent such as ethyl acetate about 65° C.

c) adding Ziprasidone hydrochloride seed compound into hot ethylacetate of step b)

d) adding formic acid solution of step a) into ethylacetate mixture of step b)

e) cooling the mixture to between about 25 to 35° C.

f) isolating the compound by filtration and washing with acetic acid and ethyl acetate

g) drying of the compound with or without vacuum at temperature of 20° C. to 130° C.

The present invention also provides a process for crystallization of Ziprasidone hydrochloride in formic acid, which includes:

a) dissolving Ziprasidone hydrochloride in formic acid;

b) cooling the reaction mass slowly;

c) isolating the product by conventional methods like filtration;

d) washing the product with organic solvents like ethyl acetate, Isopropanol, Cyclohexane etc;

e) drying of the product.

The present invention also provides process for preparing large crystals of Ziprasidone hydrochloride from Ziprasidone, base that comprises:

a) dissolving Ziprasidone base in carboxylic acid solvents like acetic acid, formic acid etc.

b) adding the hydrochloric acid digested in organic solvents like hydrochloric acid in ethyl acetate, hydrochloric acid in isopropanol etc.

c) isolating the product by conventional methods.

d) washing of the compound with organic solvents like ethyl acetate, formic acid, acetic acid, isopropanol etc.

e) drying of the compound to get ziprasidone hydrochloride having mean particle size greater than 85 micron.

Ziprasidone hydrochloride has the mean particle size or D [4,3] of greater than 85 to 300 micron.

The invention also provides formulations that include ziprasidone hydrochloride having mean particle size greater than 85 microns as ingredient, together with a carrier and, optionally, other therapeutic active ingredients. The carrier must be pharmaceutically acceptable, that is being compatible with the other ingredients of the formulation and not deleterious to the recipient. The formulation includes those suitable for oral, topical, rectal or parental administration.

Ziprasidone hydrochloride prepared by the present invention can be packed with a specific process to prevent from hydrating or to improve stability during the storage or transporting of ziprasidone hydrochloride products. This can be achieved by storing the active substance in inert atmosphere, which is technologically and economically simple. The active substance Ziprasidone HCl is stored in polythene bag or containers. The inert atmosphere may be made by nitrogen or argon or mixture thereof. Silica gel or desiccant in air permeable bag may be used optionally along with inert gas in the polythene bag or container. Inert gases may be a non-health hazardous gas, which does not react with Ziprasidone HCl and is free from moisture, which is used for packing of Ziprasidone.

The term hygroscopic active substance Ziprasidone hydrochloride or active substance Ziprasidone hydrochloride refers to Ziprasidone hydrochloride having moisture content less than 2% or Ziprasidone hydrochloride hemihydrate or anhydrous Ziprasidone hydrochloride.

Stability profile:

Stability study with normal packing (without inert atmosphere):

Initial and up to two-month stability profile with normal packing was carried out at 40° C. and 75% relative humidity conditions as follow:

| Time | Moisture content (% w/w) |
|---|---|
| Initial | 0.25% |
| One month | 3.45% |
| Two month | 4.83% |

Stability study with improved packing (inert atmosphere):

Initial and up to two-month stability profile for the hygroscopic active substance Ziprasidone HCl is shown below:

The stability studies were carried out at 40° C. and 75% relative humidity condition.

| | Moisture content | Total Impurity | Assay |
|---|---|---|---|
| Time | (% w/w) | (% w/w) | (% w/w) |
| Initial | 0.54% | 0.19% | 99.4% |
| One month | 0.41% | 0.19% | 99.4% |
| Two month | 0.54% | 0.20% | 99.7% |

The stability studies were also carried out at 25° C. and 60% relative humidity condition.

| | Moisture content | Total Impurity | Assay |
|---|---|---|---|
| Time | (% w/w) | (% w/w) | (% w/w) |
| Initial | 0.54% | 0.19% | 99.4% |
| One month | 0.38% | 0.18% | 99.2% |
| Two month | 0.48% | 0.21% | 99.3% |

X-Ray diffractograms of active substance Ziprasidone HCl (Initial and after two month sample) were recorded on Bruker Axe, DS Advance Powder X-ray Diffractometer with Cu K alpha-1 Radiation source. The characteristic X-ray diffractogram of Ziprasidone hydrochloride was found similar.

The following examples are illustrative but don't intend to limit the scope of the present invention.

### EXAMPLE 1

### Preparation of 5-(2-Chloro acetyl)-6-chloro oxindole

Cooled the reaction mixture of dichloromethane (250 ml) and aluminum chloride (159.0 gm) to a temperature of 4° C. To the cold reaction mixture 6-chloro-2-oxindole (50 gm) was added slowly over 30 minutes. The resulting reaction mixture was heated to a temperature of 26° C. To the reaction mixture chloro acetyl chloride (53.9 gm) was slowly added over 40 minutes, then refluxed the reaction mixture for 9 hours. The reaction completion was monitored by TLC. Then the reaction mass was cooled to a temperature of 28° C. and poured into a mixture of ice (900 gm) and hydrochloric acid (45 ml) and accompanied by stirring the reaction mass for 45 minutes. The separated solid was filtered and washed with water and further slurred in water (250 ml). To the resulting wet compound acetic acid (1190 ml) was added and heated to a temperature of 76° C. followed by addition of carbon (2.5 gm) to the hot reaction mass. Then the reaction mass was stirred for 15 minutes at the same temperature. Carbon was filtered off and washed with acetic acid (10 ml). Then the filtrate was cooled to a temperature of 18-20° C. and continued stirring at the same temperature for 2 hours. Then the separated solid was filtered, washed with water (2×100 ml) and dried at a temperature of 60-65° C. to afford the pure 5-(2-Chloro acetyl)-6-chloro oxindole (48.2 gm).

### EXAMPLE 2

### Preparation of 5-(2-Chloro ethyl)-6-chloro oxindole

Triethylsilane (57.2 gm) was added slowly to the reaction mixture of 5-(2-Chloro acetyl)-6-chloro oxindole (50.0 gm) and trifluoroacetic acid (175 ml) below the temperature of 45° C. Maintained the reaction at 40-45° C. for 6 hours. The reaction mass was cooled to 0 to −5° C. and maintained stirring for 90 min. The separated solid was filtered and washed with water (50 ml). Then the wet compound was further slurred in water (250 ml) for 90 min. The resultant solid was filtered, washed with water (50 ml) and dried at a temperature of 70-75° C. to afford the 5-(2-Chloro ethyl)-6-chloro oxindole (43.5 gm).

### EXAMPLE 3

### Preparation of 3-(1-Piperazinyl)-1,2-benzisothiazole

Piperazine (508.8 g) and Tert. Butanol (200 ml) were placed in round bottom flask (RBF) and then the resulting solution heated up to 90 to 100 degree C. 3-chloro-1,2-benzisothiozole (40 g) was added to the solution 5 times with in time interval of 5 to 20 minutes at the same temperature. Temperature was raised up to 110 to 130 degree C. and maintained for 16 hours. After confirmation of the completion of the reaction by TLS (Thin Layer Chromatography), the resulting solution was cooled to 80 to 90 degree C. followed by addition of water (800 ml). The above mixture was cooled to 25 to 35 degree C. and was filtered to remove the solid particles. Water (100 ml) was added to the filtrate and then pH was raised to 12-14 with caustic lye (75 ml). Toluene (400 ml) was added to the alkaline solution the resulting bi-phasic mixture was stirred vigorously for 15 to 30 minutes at 25 to 35 degree C. Organic layer separated and the aqueous layer was multiply extracted with toluene followed by combing all organic layers. The combined organic layer was washed with water (200 ml). Active carbon (10 g) was added to the washed organic layer and then filtered out the carbon. The organic solvent was evaporated till the volume reaches to 150 to 200 ml. The resulting concentrated solution was cooled to 0 to 5 degree C. and maintained for about 2 hours. Separated solids were filtered and then washed with chilled toluene (20 ml). Finally, Isolated compound was dried under reduced pressure to get titled compound 130 to 135 g.

### EXAMPLE 4

### Preparation of Ziprasidone Base (Crude)

Refluxed the reaction mixture of 5-(2-Chloro ethyl)-6-chloro oxindole (100 gm), 3-(1-piperazinyl)-1,2-benzisothiazole (104.7 gm), sodium carbonate (92.2 gm), sodium iodide (6.4 gm), tetra butyl ammonium bromide (28 gm) and cyclohexane (1000 ml) till the reaction completes. The reaction mass was cooled to a temperature of 30° C. and filtered the solid. To the wet compound added was water (1000 ml) and continued stirring for 45 minutes. The solid was filtered and washed with water (100 ml). To the water wet compound added acetone (500 ml) and stirred for 2 hours at room temperature. Filtered the compound and washed with acetone (200 ml) and dried at a temperature of 70-75° C. to afford the Crude Ziprasidone base (156.9 gm)

### EXAMPLE 5

### Preparation of Ziprasidone Base (Crude)

Charged 5-(2-Chloro ethyl)-6-chloro oxindole (50 gm), 3-(1-piperazinyl)-1,2-benzisothiazole (47.5 gm) and cyclohexane (500 ml) in to autoclave. To this sodium carbonate (46 gm), sodium iodide (3.2 gm), tetra butyl phosphonium bromide (14.8 gm) was added and maintained the reaction at temperature 95-102° C. and the pressure was 2.5 kg/cm2 till the reaction complete. The reaction mass was cooled to 30° C. and added water (250 ml), filtered the compound, washed with water (100 ml). The wet compound was further slurred in water (500 ml), filtered and washed with water (100 ml). To the water wet compound added acetone (500 ml) and stirred at room temperature for 2 hours and 30 minutes. The solid was filtered, washed with acetone (100 ml) and dried at a temperature of 60-65° C. to afford the Ziprasidone base (65.7 gm)

### EXAMPLE 6

### Purification of Ziprasidone Base

Ziprasidone base (40 gm), methanol (250 ml) and chloroform (750 ml) were heated to reflux till clear solution is obtained. Carbon (2 gm) was added to the resulting reaction solution and maintained at reflux temperature for 15 minutes. Then the carbon was filtered and the filtrate was heated to distill and collected the distillate around 400 ml. To the reaction mass added Methanol (400 ml) slowly in 45 minutes. Cooled the reaction mixture to a temperature of 35° C. and stirred at the same temperature for 2.0 hours. Then the solid was filtered, washed with methanol (200.0 ml) and dried at a temperature of 70° C. to afford the pure Ziprasidone base (31.5 gm).

### EXAMPLE 7

### Purification of Ziprasidone Base

Ziprasidone base (100 gm), methanol (1875 ml) and chloroform (625 ml) were heated to reflux till clear solution is obtained. Carbon (5 gm) was added to the resulting reaction solution and maintained at reflux temperature for 15 minutes. Then the carbon was filtered and the filtrate was heated to distill and collected the distillate around 400 ml. To the reaction mass added Methanol (1000 ml) slowly in 45 minutes. Cooled the reaction mixture to a temperature of 35° C. and stirred at the same temperature for 2.0 hours. Then the solid was filtered, washed with methanol (250.0 ml) and dried at a temperature of 70° C. to afford the pure Ziprasidone base (70-75 gm).

### EXAMPLE 8

### Preparation of Ziprasidone Hydrochloride

Dissolved the Pure Ziprasidone base (100 gm) in acetic acid (3000 ml) at temperature of 30° C. Carbon (5 gm) was added to the resulting reaction solution, stirred the reaction mixture for 15 minutes at the same temperature. Filtered carbon and washed with acetic acid (30 ml). Then add filtrate slowly to the mixer of isopropyl alcohol (500 ml) and Isopropyl alcohol-HCI (220 ml) at the temperature of 30° C. for 35 min. Stirred the reaction mixture for 2.5 hours at the same temperature. Filtered the compound and washed with Isopropyl alcohol (100 ml). To the wet compound added isopropyl alcohol (1000), stirred at 30° C. for 90 min. Then solid was filtered and washed with Isopropanol (100 ml) and dried at the temperature of 70-75° C. to afford the Ziprasidone hydrochloride of Formula (1) of the present invention (93.9 gm)

### EXAMPLE 9

### Preparation of Ziprasidone Hydrochloride

Dissolved Ziprasidone base (10 gm) in acetic acid (50 ml) at temperature of 32° C. Carbon (0.5 gm) was added to the resulting reaction solution, stirred the reaction mixture for 10 minutes at the same temperature. Then filtered carbon and washed with acetic acid (5 ml). Then added 20 ml of acetic acid-HCl slowly for 15 min to the filtrate. Stirred the reaction mixture for 90 min. filtered the separated compound and washed with acetic acid (10 ml). To the acetic acid wet added isopropyl alcohol (100), stirred at 32° C. for 60 min. Then solid was filtered and washed with Isopropanol (10 ml) and dried at the temperature of 75-80° C. to afford the Ziprasidone hydrochloride of Formula (1) of the present invention. (9.8 gm).

### EXAMPLE 10

### Preparation of Ziprasidone Hydrochloride

Ziprasidone free base (50 gm) was dissolved in acetic acid (150 ml) under nitrogen atmosphere. The resulting solution was stirred at 25-35° C. and then filtered with help of acetic acid (5 ml). The filtrate was added slowly over 45-60 minutes to a mixture of ethyl acetate (300 ml) and HCl in ethyl acetate (66 ml; HCl: EtOAc=8-12: 100 w/w) under nitrogen atmosphere. The reaction mixture was maintained at 25-35° C. for about 90-120 minutes. The resulted solids were filtered, washed with ethyl acetate (125 ml), and dried at 90-110° C. under reduced pressure to get the titled compound (49-52 gm).

### EXAMPLE 11

### Recrystallization of Ziprasidone Hydrochloride in Formic Acid

Dissolved Ziprasidone hydrochloride (50 gm) in formic acid (50 ml) at the temperature of about 85° C. Stirred the reaction mixture for 5 minutes at the same temperature. Cooled the reaction mixture to separate the compound (56° C.), then further cooled the reaction mass to 28° C. Filtered the compound and washed with ethyl acetate (2×50 ml). Dried at the temperature of 100-120° C. to afford the Ziprasidone hydrochloride (23.1 gm).

### EXAMPLE 12

### Reactive Crystallization of Ziprasidone Hydrochloride from Ziprasidone Base to get Desired Particle Size

Ziprasidone base (20 gm) was dissolved in formic acid (60 ml) at 30° C. HCl in ethyl acetate (30 ml) was added slowly over 90 min at slow stirring. The compound was filtered and washed with ethyl acetate (20 ml). The compound was dried at 110° C. to get the desired mean particle size of the Ziprasidone hydrochloride (15.2 gm). The mean particle size or D[4,3]=94.1 micron.

### EXAMPLE 13

### Crystallization of Ziprasidone Hydrochloride Using Formic Acid as Solvent and Ethyl Acetate as an Anti-Solvent

Ziprasidone hydrochloride (20 gm) was dissolved in formic acid (60 ml) at 30° C. Ethyl acetate (120 ml) was added over 15 min at slow stirring. The compound was filtered and then washed with ethyl acetate (20 ml). The compound was dried at 110-120° C. under reduced pressure to get the desired mean particle size of the Ziprasidone hydrochloride (14 gm). The mean particle size or D[4,3]=101.1 micron.

### EXAMPLE 14

### Crystallization of Ziprasidone Hydrochloride Using Formic Acid as Solvent

Ziprasidone hydrochloride (50 gm) was dissolved in formic acid (50 ml) at the temperature of about 85° C. The reaction mixture was cooled to 73° C., seeded with ziprasidone hydrochloride (0.25 gm), and maintained at 73° C. for 30 minute. Then the resulting solution was cooled to 55° C. in 30 minute for 1 hour, further cooled to 45° C. and maintained for 1 hour at 30° C. The solids were filtered, washed with ethyl acetate, dried under reduced pressure at the temperature of 30-120° C. to afford the Ziprasidone hydrochloride (23.1 gm). The mean particle size or D[4,3]=264.5 micron.

### EXAMPLE 15

### Crystallization of Ziprasidone Hydrochloride Using Formic Acid as Solvent and Ethyl Acetate as an Anti-Solvent

Ziprasidone hydrochloride (3 Kg) in formic acid (4.5 L) was heated to 60-65° C. for clear dissolution. The solution was filtered to remove any solid particles and the filtrate was cooled to 50-55° C. Ziprasidone hydrochloride (7.5 gm) as seed compound was added to the cooled filtrate. After stirred for 2 hr at 50-55° C., the resulting solution was cooled slowly to 25-35° C. over 1 to 1.5 hour. Ethyl acetate (13.5 L) was added slowly at 25-35° C. over 2 hrs. The resulting mass was stirred for 30-45 min at 25-35° C. The compound was filtered under nitrogen atmosphere. The filtered wet compound was added to acetic acid (10 L) at 25-35° C. under nitrogen atmosphere, and the resulting mixture was stirred for 30-45 min at 25-35° C. The compound was filtered under nitrogen atmosphere, washed with ethyl acetate (10 L), and then dried the compound at 60° C. under reduced pressure for 3-4 hrs and then at 110-120° C. to get the Ziprasidone hydrochloride (2.2 Kg). The product purity by HPLC was analyzed as 99.8%. The crystal size distribution as measured by Malvern was: D₁₀<18.75 micron, D₅₀=114.82 micron, D₆₀<236.5 micron and the mean particle size or D[4,3]=123.8 micron.

### EXAMPLE 16

### Crystallization of Ziprasidone Hydrochloride Using its Solution in Formic Acid as Solvent into Ethyl Acetate as an Anti-Solvent (Reserve Addition)

Charged 4.5 L of Formic acid in clean 10 L RBF at 25-35° C. and charged 1.5 Kg of ziprasidone hydrochloride under N₂ atmosphere. Stirred for few minutes for clear dissolution. Charged 13.5 L of ethyl acetate into 20 L RBF and heated to 65° C. Added 30 gms of Ziprasidone hydrochloride seed into the ethyl acetate and stirred for 10-15 minutes. Added formic acid solution to ethyl acetate mixture at 60-65° C. in 2½-3½ hr (kept N₂ atmosphere in RBF). Cooled the solution to 30° C. in 2-3 hr. Filtered the product and washed the wet cake with 6 L of acetic acid. Finally washed with 6 L of ethyl acetate. Dried the product at 90° C. under vacuum till constant weight is obtained. The mean particle size or D[4,3]=156.2 micron.

### EXAMPLE 17

### Crystallization of Ziprasidone Hydrochloride Using its Solution in Formic Acid as Solvent into Ethyl Acetate as an Anti-Solvent (Reserve Addition)

Charged 60 L of Formic acid in clean 630 L reactor at 25-35° C. and charged 20 Kg of ziprasidone hydrochloride under N₂ atmosphere. Stirred for few minutes till clear dissolution. Charged 180 L of ethyl acetate into another 630 L reactor and heated to 65° C. Added 400 gms of Ziprasidone hydrochloride as seed into the ethyl acetate and stirred for 10-15 minutes. Added formic acid solution to ethyl acetate mixture at 60-65° C. in 2½-3½ hr (kept N₂ atmosphere). Cooled the solution to 30° C. in 2-3 hr. Filtered the product and washed the wet cake with 80 L of acetic acid. Finally washed with 80 L of ethyl acetate. Dried the product at about 95° C. under vacuum till constant weight is obtained. The product purity by HPLC was analyzed as 99.7%. D[4,3]=132.2 micron.

1. A process for the preparation of 5-(2-Chloro acetyl)-6-chloro oxindole of Formula (IV), which is an intermediate for the preparation of 5-(2-chloro ethyl)-6-chloro oxindole of Formula (V) comprises:
i. refluxing the reaction mixture of 6-chloro-2-oxindole of Formula (II) and chloro acetyl chloride of Formula (III) in presence of halogenated hydrocarbon solvent including dichloromethane, chloroform, ethylene dichloride, carbon tetrachloride and Lewis acids including AlCl₃, BF₃. (Et₂O)₂, as catalyst till the reaction completes;
ii. cooling the reaction mass obtained in step (i) and hydrolysed into chilled water at a temperature of 15-45° C., accompanied by stirring the reaction mass at the same temperature till the solid separates;
iii. successive filtering and washing the compound 5-(2-Chloro acetyl)-6-chloro oxindole obtained in step (ii) with water;
iv. purifying 5-(2-Chloro acetyl)-6-chloro oxindole obtained by step (iii) or by different process in organic acids including acetic acid, formic acid, propionic acid by the process which comprises adding organic acid followed by heating the reaction mixture to a temperature of 60-100° C.;
v. optionally adding activated carbon to the reaction mass of step (iv) and stirring at same temperature;
vi. filtering the carbon from reaction mass obtained in step (v);
vii. maintaining the filtrate obtained in step (vi) to a temperature of 10-40° C.;
viii. filtering and washing the solid obtained in step (vii) with water;
ix. drying the solid obtained step (viii) to a temperature of 50-100° C., to afford pure 5-(2-Chloro acetyl)-6-chloro oxindole.

2. An improved process for the preparation of 5-(2-(4-(1,2-benzisothiazol-3-yl)-1-piperazinyl) ethyl)-6-chloro-1,3-dihydro-2H-indol-2-one (Ziprasidone Base) of Formula (VII) comprises;
i. carrying out the reaction between the 3-(1-piperazinyl)-1,2-benzisothiazole of Formula (VI), 5-(2-chloro ethyl)-6-chloro oxindole of Formula (V), sodium carbonate and sodium iodide in a hydrocarbon solvents including Cyclohexane, toluene, benzene, hexanes, heptane preferably Cyclohexane in presence of a phase transfer catalyst comprises of tetrabutyl ammonium bromide, tetrabutyl phosphonim bromide, tetraethyl ammonium bromide, tetrabutyl ammonium iodide preferably tetrabutyl ammonium bromide till the reaction completes at reflux or pressure conditions;
ii. cooling the reaction mass obtained in step (i) to a temperature of 15-40° C.;
iii. filtering the compound obtained in step (ii);
iv. adding water to the wet compound obtained in step (iii) accompanied by stirring;
v. washing the compound obtained in step (iv) with water;
vi. suspending the compound obtained in step (v) in alkanone solvents including acetone, 2-butanone, propanone, methylethyl ketone, dimethyl ketone, diethyl ketone, preferably acetone accompanied by stirring;
vii. isolating the solid obtained in step (vi);
viii. drying the isolated compound of step (vii) at 70 to 75° C. to afford the Ziprasidone base of Formula (VII).

3. Process for the purification of Ziprasidone Base of Formula (VII), prepared according to the claim 2 comprises of:
i. dissolving the Ziprasidone Base of Formula (VII) in mixture of aliphatic alcoholic solvents including methanol, ethanol, propanol, butanol, isopropanol, isobutanol, preferably methanol and halogenated hydrocarbon solvents including methylene chloride, chloroform, ethylene dichloride, carbon tetrachloride, preferably chloroform at reflux temperature;
ii. charging carbon to the reaction solution of step (i) and followed by maintaining at reflux condition for 10-15 minutes;
iii. filtering the carbon from reaction mass obtained in step (ii);
iv. optionally distilling off the solvent from the filtrate of step (iii)to about half to one third of the volume;
v. adding alcoholic solvents including methanol, ethanol, propanol, butanol, isopropanol, isobutanol, preferably methanol to the residue of step (iv) at a temperature of 20-60° C. for 30 minutes, preferably 10-15 minutes;
vi. cooling the reaction mass of step (v) to 10 to 35° C., accompanied by maintaining the reaction mass at the same temperature till the solid separates out;
vii. filtering and washing the solid obtained in step (vi) with alcoholic solvents including methanol, ethanol, propanol, butanol, isopropanol, isobutanol, preferably methanol;
viii. drying the isolated compound of step (vii) at 50-100° C.; to afford pure ziprasidone base of Formula (VIII).

4. Process for the preparation of Ziprasidone hydrochloride of Formula (I) from ziprasidone base of Formula (VIII), prepared according to the claim 3 comprises;
i. dissolving Ziprasidone base of Formula (VIII) in organic acids including acetic acid, formic acid, preferably acetic acid or a mixture of organic acids and alcohols at room temperature;
ii. charging carbon to the reaction solution of step (i) accompanied by stirring for 30 minutes;
iii. filtering the carbon from reaction mass obtained in step (ii) accompanied by adding hydrochloric acid in organic solvents including acetic acid, isopropanol , methanol, preferably HCl in acetic acid or HCl in isopropanol to the filtrate or alternatively adding the solution obtained step (ii) to the mixture of hydrochloric acid in organic solvents like acetic acid, Isopropanol or mixture of acetic acid and isopropanol;
iv. maintaining the reaction mixture at 30-35° C. till the solid separates out;
v. isolating the solid obtained in step (iv);
vi. optionally slurrying the isolated compound from alcoholic solvents including methanol, ethanol, propanol, butanol, isopropanol, isobutanol, preferably isopropanol;
vii. drying the solid obtained in step (vi) at 50-100° C., to afford ziprasidone hydrochloride of Formula (I).

5. The process according to claim 1 of step (i), wherein the halogenated hydrocarbon solvent is dichloromethane.

6. The process according to claim 1 of step (iv), wherein the organic acid used is acetic acid.

7. The process according to claim 2 of step (i), wherein the hydrocarbon solvent is cyclohexane.

8. The process according to claim 2 of step (i), where in the phase transfer catalyst is tetrabutyl ammonium bromide.

9. The process according to claim 2 of step (vi), where in the alkanone solvent is acetone.

10. The process according to claim 2 of step (vii), where in the isolating solvent is acetone.

11. The process according to claim 3 of step (i), where in the mixture of alcoholic and halogenated hydrocarbon solvents used in which alcohols are methanol, ethanol, propanol, butanol, isopropanol, isobutanol etc, and halogenated hydrocarbons are methylene chloride, chloroform, ethylene dichloride, carbontetrachloride.

12. The process according to claim 11, where in the alcoholic solvent is methanol.

13. The process according to claim 11, where in the haogenated hydrocarbon is chloroform.

14. The process according to claim 3 of step (vii), where in the alcoholic solvent is methanol.

15. The process according to claim 4 of step (i), where in the organic acid is acetic acid.

16. The process according to claim 4 of step (iii), where in the acidifying agent is HCl in acetic acid.

17. The process according to claim 4 of step (vi), where in the solvent is used for slurrying is isopropanol.

18. Ziprasidone hydrochloride having a mean particle size which is greater than about 85 micron and less than about 300 micron.

19. A process for crystallizing ziprasidone hydrochloride comprising dissolving ziprasidone hydrochloride in formic acid and crystallizing ziprasidone hydrochloride.

20. The process of claim 19, wherein said crystallization is performed by adding the solution of ziprasidone into an anti-solvent.

21. The process of claim 20, wherein the anti-solvent is ethyl acetate.

22. The process of claim 20, wherein the anti-solvent contains a seed of ziprasidone hydrochloride.

23. A pharmaceutical composition comprising ziprasidone hydrochloride having a mean particle size of greater than about 85 micron and less than about 300 micron and a pharmaceutically acceptable carrier.
